(19) United States
(12) Patent Application Publication
(10) Pub. No.: US 2013/0103111 A1
(43) Pub. Date: Apr. 25, 2013

(54) FREQUENCY-TO-DIGITAL CONVERSION-BASED TRANSCUTANEOUS TRANSMISSION

(71) Applicant: Werner Meskens, Opwijk (BE)

(72) Inventor: Werner Meskens, Opwijk (BE)

(21) Appl. No.: 13/277,807

(22) Filed: Oct. 20, 2011

(51) Int. Cl. A61F 11/04

(52) U.S. Cl. 607/57

(57) ABSTRACT

A method for use in an active implantable medical device (AIMD) including an external module and an implantable module having a stimulation transducer implantable in an implantee and configured to deliver stimulation energy to auditory tissue so as to cause a hearing percept, the method including: receiving, at the implantable module, from the external module via a transcutaneous RF link, an analog frequency-modulated RF signal (analog FM) including stimulation signals representative of sound; performing frequency-to-digital conversion upon the frequency-modulated signal to obtain pulse-formatted signals corresponding to the stimulation signals; and energizing the stimulation transducer based upon the pulse-formatted signals to cause the hearing percept.

### BACKGROUND

1. Field of the Present invention

The present invention relates generally to transcutaneous signal transmission (TST) systems for Active Implantable Medical Devices (AIMDs), and more particularly to such systems using frequency-to-digital conversion.

2. Related Art

A variety of medical implants exist to assist (e.g., via neurostimulation) people who suffer diminished capability of one or more senses (e.g., sight or hearing) and/or one or more other physiological processes.

Implantable medical devices have one or more components or elements that are at least partially implantable in a recipient. One type of implantable medical device is an active implantable medical device (AIMD), which is a medical device having one or more implantable components, the latter being defined as relying for its functioning upon a source of power other than the human body or gravity, such as an electrical energy source. Exemplary AIMDs include devices configured to provide one or more of stimulation and sensing, such as implantable stimulator systems and implantable sensor systems. Exemplary implantable sensor systems include, but are not limited to, sensor systems configured to monitor cardiac, nerve and muscular activity.

Implantable stimulator systems provide stimulation to the implantee. Exemplary implantable stimulator systems include, but are not limited to, cochlear implants, auditory brain stem implants, bone conduction devices, cardiac pacemakers, neurostimulators, functional electrical stimulation (FES) systems, etc. A cardiac pacemaker is a medical device that uses electrical impulses, delivered by electrodes contacting the heart muscles, to regulate the beating of a heart. The primary purpose of a pacemaker is to maintain an adequate heart rate. A neurostimulator, also sometimes referred to as an implanted pulse generator (IPG), is designed to deliver electrical stimulation to the brain. Neurostimulators are sometimes used for deep brain stimulation and vagus nerve stimulation to treat neurological disorders. FES systems use electrical currents to activate nerves innervating extremities affected by paralysis resulting from, e.g., spinal cord injury, head injury, stroke, or other neurological disorders. Other types of implantable stimulator systems include systems configured to provide electrical muscle stimulation (EMS), also known as neuromuscular stimulation (NMES) or electromyostimulation, which involves the application of electric impulses to elicit muscle contraction.

People who suffer from a loss of hearing may be assisted by various devices including some types of medical implants. One such device is a hearing aid, which amplifies and/or clarifies surrounding sounds and directs this into the person's ear. Another device is a cochlear implant, which is used to treat sensorineural hearing loss by providing electrical energy directly to the implantee's auditory nerves via an electrode assembly implanted in the cochlea. Electrical stimulation signals are delivered directly to the auditory nerve via the electrode assembly, thereby inducing a hearing sensation in the implant recipient.

If a person's cochlea is functioning well but his middle ear is defective, another type of hearing device that may be used is a mechanical actuator type which provides direct mechanical vibrations to a part of the person's hearing system such as the middle ear, inner ear, or bone surrounding the hearing system. One variety of mechanical actuator type hearing device is referred to as a Direct Acoustic Cochlear Stimulation (DACS) system, in which the actuator operates directly on the cochlea.

Another type of implantable hearing device is an Auditory Brain Stem Implant (ABI) device. ABIs are typically used in recipients suffering from sensorineural hearing loss and who, due to damage to the recipient's cochlea or auditory nerve, are unable to use a cochlear implant. Yet another type of implantable hearing device is referred to as a bone conduction system, and it converts a received sound into mechanical vibrations. The vibrations are transferred through the skull to the cochlea causing generation of nerve impulses, which result in the perception of the received sound. Bone conduction devices may be a suitable alternative for individuals who cannot derive sufficient benefit from acoustic hearing aids, cochlear implants, etc.

In more detail, a DACS system includes an external part that receives and processes surrounding acoustic energy, and then transmits control signals to an implantable part based upon the acoustic energy. The external part transforms the acoustic energy into data and converts the data into radio frequency (RF) signals that can be transmitted wirelessly through the skin of the implantee (i.e., transmitted transcutaneously) via a transmitting circuit and a coil in the external part. The internal, implanted part includes a coil, a receiving circuit for receiving the transmitted RF signals and converting the same into control signals, and an actuator to receive the control signals and transform the same into movement. By such movement, the actuator acts directly upon a part of the implantee's hearing system such as a part of the inner ear (e.g. the stapes) or directly upon the oval window of the cochlea. Such movement generates vibrations in the cochlear fluid that stimulate hair cells. In response, the hair cells stimulate nerves connected directly to the brain, with such nervous stimulation being perceived as sound.

The implantable part requires power to operate. In some types of DACS systems, the power is provided by a discrete physical connection to local, e.g., implanted power supply. In other systems, the power may be provided via a transcutaneous power link transferred, e.g., wirelessly between external and implantable coils.

FIG. 1 illustrates an example of a medical implant system 100, e.g., a DACS system, according to the Background Art to which various aspects described herein may be applied.

FIG. 1 illustrates, according to the Background Art, a medical implant system 100 including an external module 10 and an implantable module 20. In FIG. 1, the external module 10 includes: an audio source and/or a microphone 12; a power source 16; a signal pre-processing block 17 (e.g., a conditioning amplifier); a first pulse modulator 13 (e.g., a pulse width modulation (PWM) modulator or a pulse density (PDM) modulator; a digital, second pulse modulator (upconverter) (e.g., a frequency shift keying (FSK) modulator) 14; an RF driver 15; and a transmitting antenna system (e.g., a coil) 11. A transmission signal from the digital, second pulse modulator 14 is amplified by the RF driver 15 and then the amplified signal is applied to the coil 11 for wireless transmission transcutaneously via a layer of skin 50 to the implanted implantable module 20.

In FIG. 1, the implantable module 20 includes: a receiving antenna system (e.g., an implantable coil) 21; a power & modulation extractor unit 24 that itself includes a rectification unit 24a (e.g., a diode-based circuit configured to provide half-wave or full-wave rectification); a power storage device 30 (e.g., as a capacitor or small battery); an FSK demodulator (downconverter) 25; a driver/amplifier 26; an integrator 28, e.g., a low pass filter (LPF); a load-matching block 29; and an actuator. The modulated signal transferred wirelessly from the external module 10 is received by the implantable coil 21 and forwarded to the power and modulation extracting block 24, which extracts power from the modulated signal for powering (among others) the demodulator 25 and the driver/amplifier 26 and also transfers the modulated signal to the demodulator 25. Optionally, the implantable module 20 may also include an audio pre-processing block (not illustrated in FIG. 1) for improving or optimizing the audio signal quality prior to demodulation and/or post-processing circuitry (not illustrated in FIG. 1).

In FIG. 1, the received modulated signal also is processed to extract control information or control signals to actuate the mechanical actuator 23. More particularly, the received modulated signal is applied to the input of the FSK demodulator 25, which removes the FSK modulation that had been applied by the external module 10. This FSK demodulated signal is then applied directly to the driver/amplifier 26, e.g., a class D amplifier. The amplified output of the amplifier 26 is then applied to the LPF 28, the output of which is adaptively or optimally load-matched to an impedance of the actuator 23 by the load-matching block 29. Depending on the type of actuator load, the matching block 29 and low-pass filter 28 could be implemented by a single block with combined functionality, e.g., a passive network of inductors and/or capacitors. The output of the load-matching block 29 is then applied to the mechanical actuator 23 which generates stimulating vibrations in accordance with the signals applied.

FIG. 2 illustrates, according to the Background Art, back-end components 200 an implantable module 20 of a DACS system, e.g., as in FIG. 1. In FIG. 2, the following is noted: amplifier 26 is illustrated as a Class D amplifier that includes complimentary MOSFETs configured in a push-pull arrangement; and the integrator 28 and the load-matching block 29 are illustrated as a second order low pass filter (LPF) that also exhibits a load-matching function. Also in FIG. 2, an end 23b of actuator 23 is connected to stapes (not illustrated in FIG. 2) of the implantee's middle ear.

FIG. 3 illustrates, according to the Background Art, an example implementation of the RF driver 15 of FIG. 1 in the context of the medical implant system 100 being a bone conduction system. In FIG. 3, the RF driver 15 is configured with a differential output. A primary coil L is tuned, e.g., to about 5 MHz resonance by a series capacitor C (e.g., 47 pF in parallel with 7-100 pF). Inverter gates of differential output drivers are placed 2 by 2 in parallel to provide sufficient current going through the series resonant circuit LC. In the example of FIG. 3, the RF driver 15 includes a total of six inverter logic gates (e.g., IC 74AC04). Also, e.g., four diodes (e.g., MCL4148) are included to protect the RF driver 15 from high transients caused by the LC tank or electrostatic discharge (ESD).

An alternative to traditional delta-sigma (Δ-Σ) modulation (DSM) is frequency DSM (FDSM). A traditional DSM includes an integrator. In an FDSM, the integrator of the traditional DSM is replaced with a frequency modulator.

### SUMMARY

In one aspect of the present invention, there is provided a method, for use in an active implantable medical device (AIMD) including an external module and an implantable module having a stimulation transducer implantable in an implantee and configured to deliver stimulation energy to auditory tissue so as to cause a hearing percept, the method comprising: receiving, at the implantable module, from the external module via a transcutaneous RF link, an analog frequency-modulated RF signal (analog FM) including stimulation signals representative of sound; performing frequency-to-digital conversion upon the frequency-modulated signal to obtain pulse-formatted signals corresponding to the stimulation signals; and energizing the stimulation transducer based upon the pulse-formatted signals to cause the hearing percept.

In another aspect, there is provided an implantable module of an active implantable medical device (AIMD) implantable in an implantee, the implantable module comprising: an antenna to receive an analog frequency-modulated signal including stimulation signals representative of sound, a frequency-to-digital converter operable upon the frequency-modulated signal to obtain pulse-modulated signals; a driver circuit responsive to the frequency-to-digital converter; and a stimulation transducer responsive to the driver circuit; the driver circuit being configured to energize the stimulation transducer based upon the pulse-formatted signals; and the stimulation transducer being configured to deliver stimulation energy to auditory tissue based upon stimulation signals so as to cause a hearing percept.

In yet another aspect, there is provided an active implantable medical device (AIMD) including an external module and an implantable module having a stimulation transducer implantable in an implantee and configured to deliver stimulation energy to auditory tissue so as to cause a hearing percept, the method comprising: performing, at the external module, analog frequency-modulation (analog FM) upon sound signals; receiving, at the implantable module, from the external module via a transcutaneous RF link, a frequency-modulated RF signal including stimulation signals representative of sound; performing frequency-to-digital conversion upon the frequency-modulated signal to obtain pulse-formatted signals corresponding to the stimulation signals; and energizing the stimulation transducer based upon the pulse-formatted signals to cause the hearing percept; and wherein, taken together, the frequency modulation and the frequency-to-digital conversion represent a distributed form of frequency delta-sigma (FDS) modulation (FDSM).

In yet another aspect, there is provided an implantable module of an active implantable medical device (AIMD) implantable in an implantee, the implantable module comprising: an analog frequency-modulation modulator to produce frequency-modulated signals representing sound signals; a first antenna to transmit a radio frequency (RF) signal including the frequency-modulated signals; a second antenna to receive a frequency-modulated RF signal; a frequency-to-digital converter operable upon the frequency-modulated RF signal to obtain pulse-formatted signals; a driver circuit responsive to the frequency-to-digital converter; and a stimulation transducer responsive to the driver circuit; the driver circuit being configured to energize the stimulation transducer based upon the pulse-formatted signals; and the stimulation transducer being configured to deliver stimulation energy to auditory tissue based upon stimulation signals so as to cause a hearing percept; and. wherein, taken together, the frequency modulation and the frequency-to-digital conversion represent a distributed form of frequency delta-sigma (FDS) modulation (FDSM).

### BRIEF DESCRIPTION OF THE DRAWINGS

Illustrative embodiments of the present invention are described herein with reference to the accompanying drawings, in which:

FIG. 1 illustrates an example of a medical implant system, according to the Background Art;

FIG. 2 illustrates, according to the Background Art, back-end components in an implantable module of, e.g., the medical implant system as in FIG. 1;

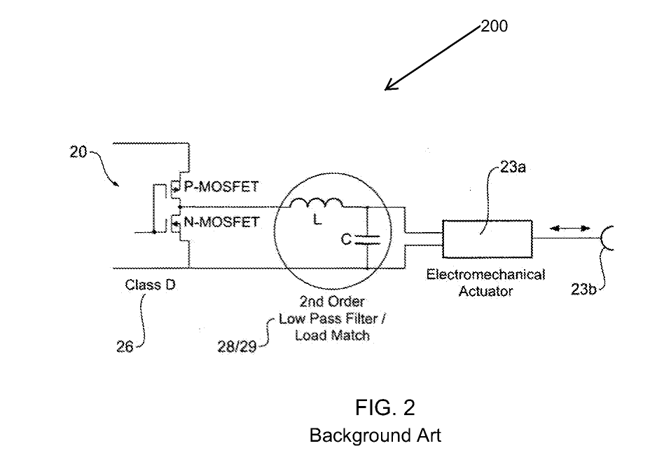

FIG. 3 illustrates, according to the Background Art, an example implementation of circuit for the RF driver, e.g., as in FIG. 1;

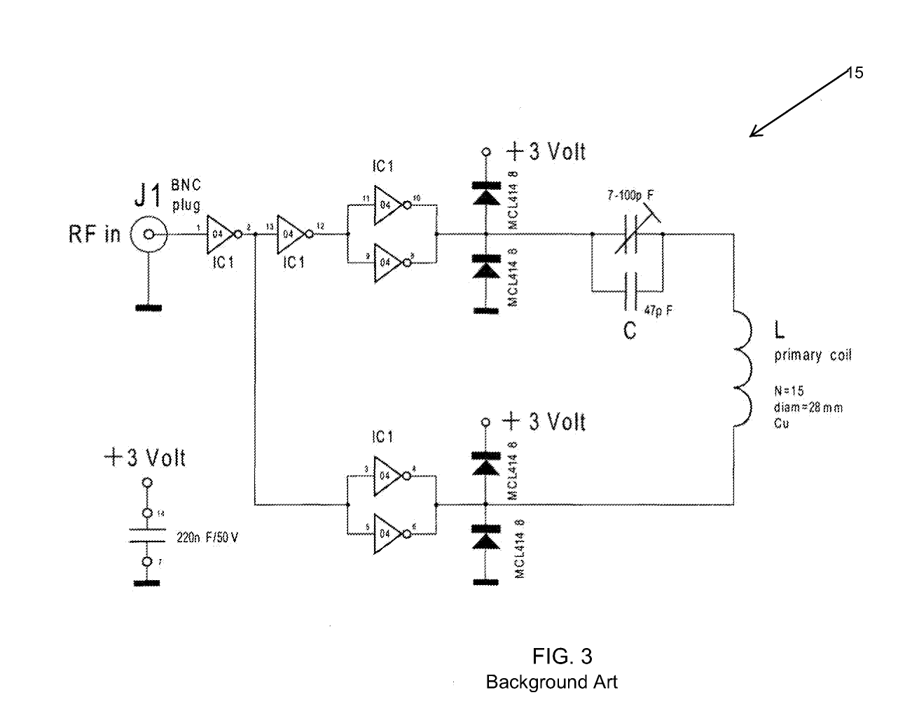

FIG. 4 is perspective view of an individual's head in which an auditory prosthesis in accordance with embodiments of the present invention may be implemented;

FIG. 5A is a perspective view of an exemplary DACS, in accordance with embodiments of the present invention;

FIG. 5B is a perspective view of another type of DACS, in accordance with an embodiment of the present invention;

FIG. 6 illustrates an example of a medical implant system, e.g., a DACS system, a bone conduction system, a cochlear implant system, etc., according to an embodiment of the present invention;

FIG. 7 illustrates details of an example of a frequency-to-digital converter, according to an embodiment of the present invention;

FIG. 8 illustrates details of another example of a frequency-to-digital converter, according to an embodiment of the present invention; and

FIG. 9 illustrates details of illustrates an example of a medical implant system, e.g., a bone conduction system, according to an embodiment of the present invention;.

### DETAILED DESCRIPTION

Embodiments of the present invention are generally directed to transcutaneous frequency delta-sigma modulation in an active implantable medical device (AIMD).

An active implantable medical device (AIMD) can include an external module and an implantable module having a stimulation transducer implantable in an implantee and configured to deliver stimulation energy to auditory tissue so as to cause a hearing percept. At the implantable module, a frequency-modulated RF signal including stimulation signals representative of sound is received via a transcutaneous RF link. Next, frequency-to-digital conversion is performed upon the frequency-modulated signal to obtain pulse-formatted signals corresponding to the stimulation signals. Then the stimulation transducer is energized based upon the pulse-formatted signals to cause the hearing percept.

The external module performs frequency modulation up sound signals, which are then modified to be RF signals and then transferred via the transcutaneous link. Taken together, frequency modulation and the frequency to digital conversion represent a distributed form of frequency delta-sigma (FDS) modulation (FDSM).

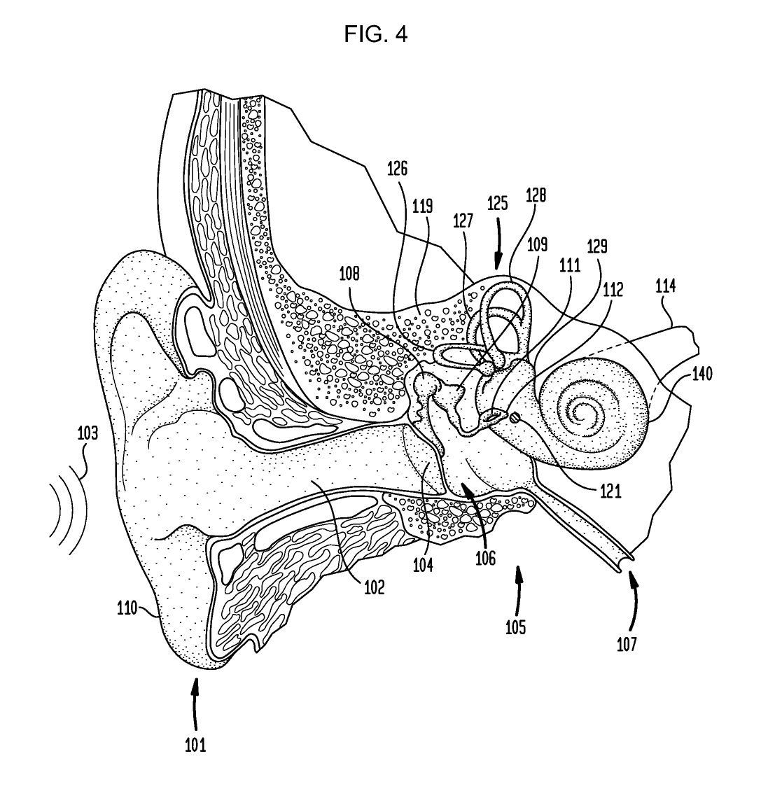

FIG. 4 is perspective view of an individual's head in which an auditory prosthesis in accordance with embodiments of the present invention may be implemented. As shown in FIG. 4, the individual's hearing system comprises an outer ear 101, a middle ear 105 and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound. Also, there are semicircular canals 125, namely horizontal semicircular canal 126, posterior semicircular canal 127, and superior semicircular canal 128.

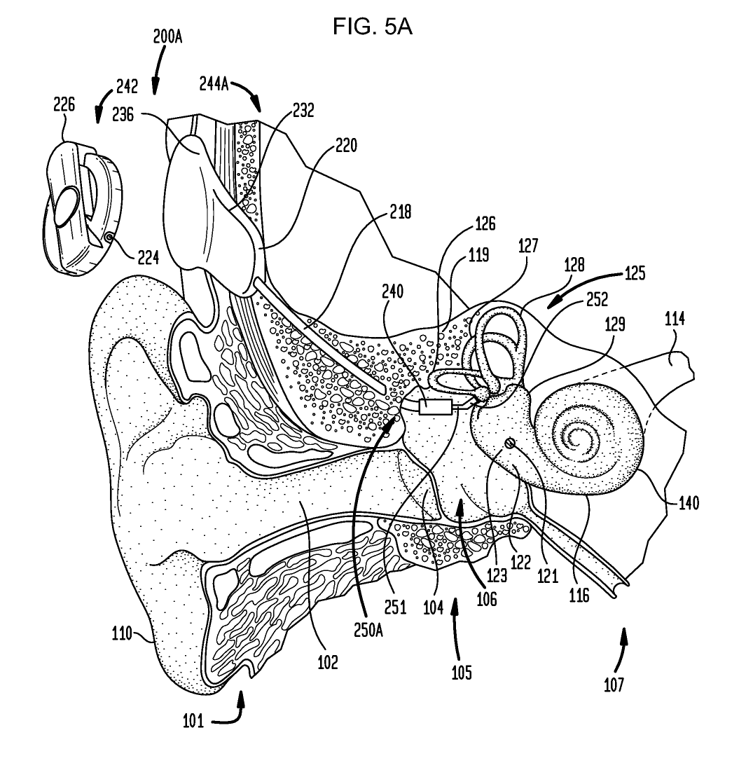

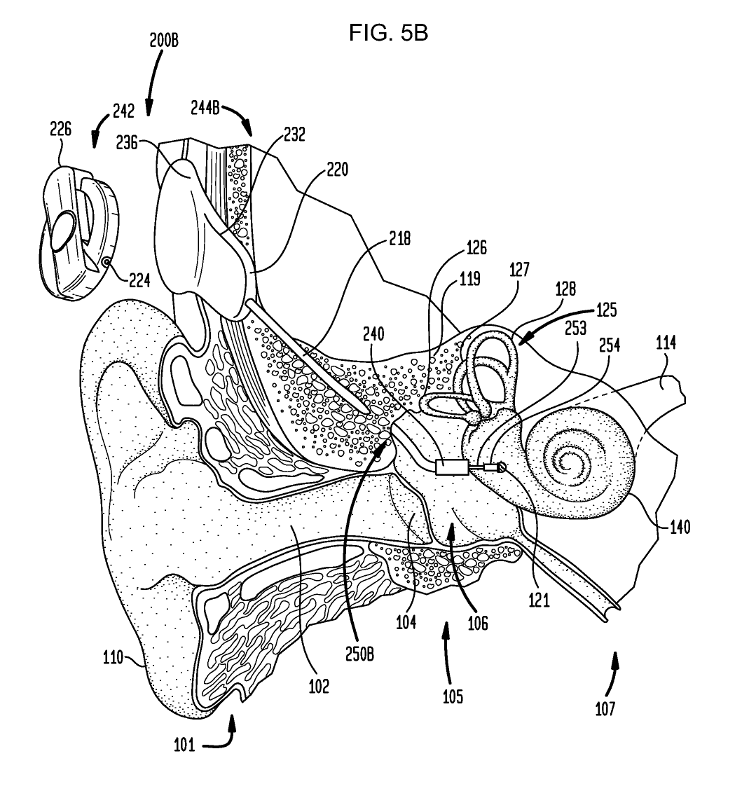

One type of auditory prosthesis that converts sound to mechanical stimulation in treating hearing loss is a direct acoustic cochlear stimulator (DACS) (also sometimes referred to as an “inner ear mechanical stimulation device” or “direct mechanical stimulator”). A DACS generates vibrations that are directly coupled to the inner ear of a recipient and thus bypasses the outer and middle ear of the recipient. FIG. 5A is a perspective view of an exemplary DACS 200A in accordance with embodiments of the present invention.

DACS 200A comprises an external component 242 that is directly or indirectly attached to the body of the recipient, and an internal component 244A that is temporarily or permanently implanted in the recipient. External component 242 typically comprises one or more sound input elements, such as microphones 224 for detecting sound, a sound processing unit 226, a power source (not shown), and an external transmitter unit (also not shown). The external transmitter unit is disposed on the exterior surface of sound processing unit 226 and comprises an external coil (not shown). Sound processing unit 226 processes the output of microphones 224 and generates encoded signals, sometimes referred to herein as encoded data signals, which are provided to the external transmitter unit. For ease of illustration, sound processing unit 226 is shown detached from the recipient.

Internal component 244A comprises an internal receiver unit 232, a stimulator unit 220, and a stimulation arrangement 250A. Internal receiver unit 232 and stimulator unit 220 are hermetically sealed within a biocompatible housing, sometimes collectively referred to herein as a stimulator/receiver unit.

Internal receiver unit 232 comprises an internal coil (not shown), and preferably, a magnet (also not shown) fixed relative to the internal coil. The external coil transmits electrical signals (i.e., power and stimulation data) to the internal coil via a radio frequency (RF) link. The internal coil is typically a coil, e.g., a wire loop antenna comprised of multiple turns of electrically insulated single-strand or multi-strand platinum or gold wire. The electrical insulation of the internal coil is provided by a flexible silicone molding (not shown). In use, implantable receiver unit 232 is positioned in a recess of the temporal bone adjacent auricle 110 of the recipient in the illustrated embodiment.

In the illustrative embodiment, stimulation arrangement 250A is implanted in middle ear 105. For ease of illustration, ossicles 106 have been omitted from FIG. 5A. However, it should be appreciated that stimulation arrangement 250A is implanted without disturbing ossicles 106 in the illustrated embodiment.

Stimulation arrangement 250A comprises an actuator 240, a stapes prosthesis 252 and a coupling element 251. In this embodiment, stimulation arrangement 250A is implanted and/or configured such that a portion of stapes prosthesis 252 abuts an opening in one of semicircular canals 125. For example, in the illustrative embodiment, stapes prosthesis 252 abuts an opening in horizontal semicircular canal 126. It would be appreciated that in alternative embodiments, stimulation arrangement 250A is implanted such that stapes prosthesis 252 abuts an opening in posterior semicircular canal 127 or superior semicircular canal 128.

As noted above, a sound signal is received by one or more microphones 224, processed by sound processing unit 226, and transmitted as encoded data signals to internal receiver 232. Based on these received signals, stimulator unit 220 generates drive signals which cause actuation of actuator 240. This actuation is transferred to stapes prosthesis 252 such that a wave of fluid motion is generated in horizontal semicircular canal 126. Because, vestibule 129 provides fluid communication between the semicircular canals 125 and the median canal, the wave of fluid motion continues into median canal, thereby activating the hair cells of the organ of Corti. Activation of the hair cells causes appropriate nerve impulses to be generated and transferred through the spiral ganglion cells (not shown) and auditory nerve 114 to the brain (also not shown) where they are perceived as sound.

FIG. 5B is a perspective view of another type of DACS 200B in accordance with an embodiment of the present invention. DACS 200B comprises an external component 242 which is directly or indirectly attached to the body of the recipient, and an internal component 244B which is temporarily or permanently implanted in the recipient. As described above with reference to FIG. 5A, external component 242 typically comprises one or more sound input elements, such as microphones 224, a sound processing unit 226, a power source (not shown), and an external transmitter unit (also not shown). Also as described above, internal component 244B comprises an internal receiver unit 232, a stimulator unit 220, and a stimulation arrangement 250B.

In the illustrative embodiment, stimulation arrangement 250B is implanted in middle ear 105. For ease of illustration, ossicles 106 have been omitted from FIG. 5B. However, it should be appreciated that stimulation arrangement 250B is implanted without disturbing ossicles 106 in the illustrated embodiment.

Stimulation arrangement 250B comprises an actuator 240, a stapes prosthesis 254 and a coupling element 253 connecting the actuator to the stapes prosthesis. In this embodiment stimulation arrangement 250B is implanted and/or configured such that a portion of stapes prosthesis 254 abuts round window 121.

As noted above, a sound signal is received by one or more microphones 224, processed by sound processing unit 226, and transmitted as encoded data signals to internal receiver 232. Based on these received signals, stimulator unit 220 generates drive signals which cause actuation of actuator 240. This actuation is transferred to stapes prosthesis 254 such that a wave of fluid motion is generated in the perilymph in scala tympani. Such fluid motion, in turn, activates the hair cells of the organ of Corti. Activation of the hair cells causes appropriate nerve impulses to be generated and transferred through the spiral ganglion cells (not shown) and auditory nerve 114 to the brain (also not shown) where they are perceived as sound.

It should be noted that the embodiments of FIGS. 5A and 5B are but two exemplary embodiments of a DACS, and in other embodiments other types of DACs are implemented. Further, although FIGS. 5A and 5B provide illustrative examples of a DACS system, in embodiments a middle ear mechanical stimulation device can be configured in a similar manner, with the exception that instead of the actuator 240 being coupled to the inner ear of the recipient, the actuator is coupled to the middle ear of the recipient. For example, in an embodiment, the actuator stimulates the middle ear by direct mechanical coupling via coupling element to ossicles 106, such as to incus 109.

In determining the drive signals to cause actuation of actuator 240, the resonance peak of the actuator are be taken into account by the stimulator unit 220 in the presently described embodiment. Resonance refers to the tendency of a system to oscillate with a larger amplitude at some frequencies than at others. And, a resonance peak refers to frequencies at which a peak in the amplitude occurs.

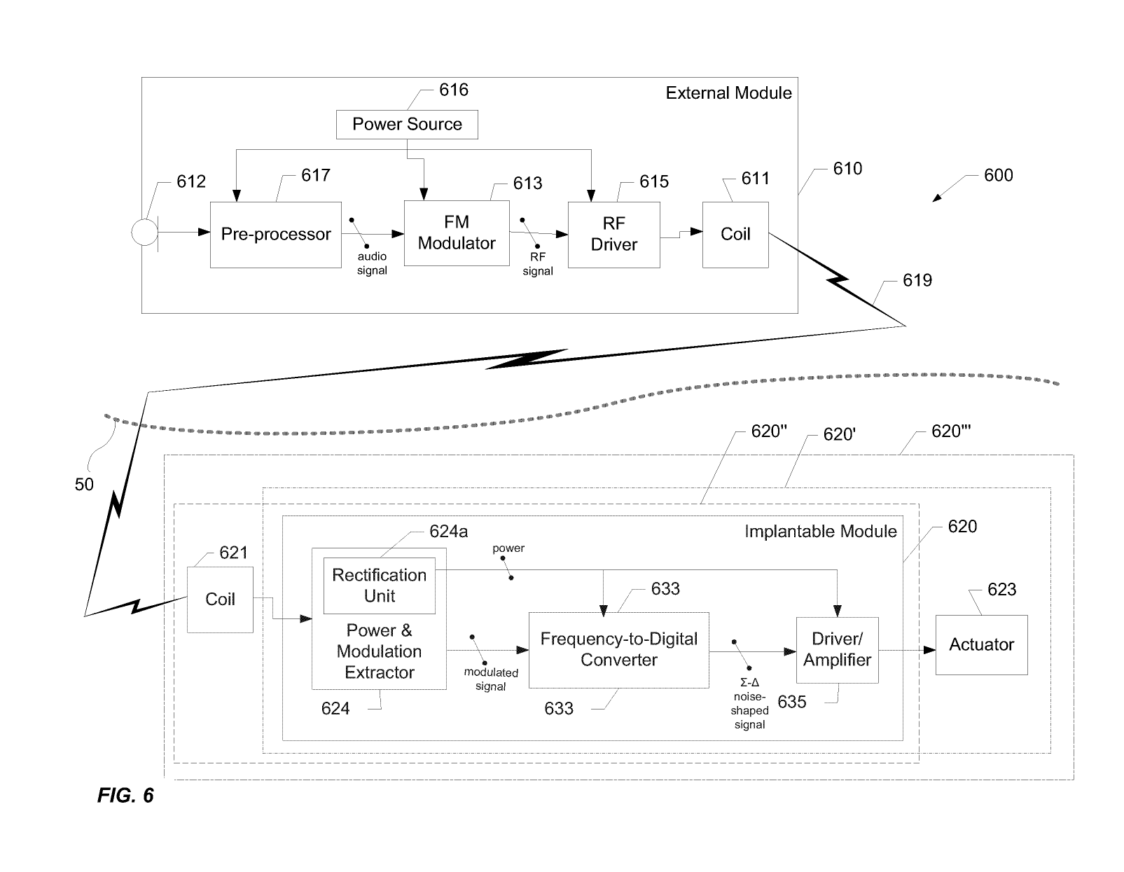

FIG. 6 illustrates an example of a medical implant system 600, e.g., a DACS (again, Direct Acoustic Cochlear Stimulation) system, a bone conduction system, a cochlear implant system, etc., according to an embodiment of the present invention. The system 600 includes an external module 610 and an implantable module 620, the latter having been implanted into an implantee as indicated via a layer of skin 50 of the implantee's body (not illustrated in FIG. 6), e.g., a portion of the implantee's scalp. The external module 610 is operable to transfer signals wirelessly 619 and transcutaneously through the layer 50 of tissue of the implantee to the implantable module 620.

As illustrated in FIG. 6, the medical implant system 600 further includes a stimulation transducer 623, e.g., an actuator such as a piezoelectric actuator, that is not included within a housing 620 of the implantable module 620. In other embodiments, the actuator 623 may be provided within the housing of the implantable module, e.g., as indicated by the phantom boxes 620′ and 620′″. As will be discussed further below, the wireless, transcutaneous transmission (RF link) can be achieved by inductively coupled coils. Also, e.g., the implantable module can be implemented using an ASIC (application specific integrated circuit).

During the development of the present invention, among other things, the inventor contemplated the following design factors: because the RF transcutaneous link between the external module and the implantable module should transfer power efficiently from external module to the implantable module, the Q-factor of each of the inductively coupled coils in resonance should be relatively high; and, on the other hand, higher Q-factors limit bandwidth and decrease integrity of the information in the transferred signal.

Also during the development of the present invention, among other things, the inventor contemplated the following: the presence of a layer (e.g., 50 in FIG. 6) of tissue in a communication channel between a coil (611) from a primary LC-resonant tank of an external module (e.g., 610 in FIG. 6) and a coil (621) from a secondary LC-resonant tank of an implantable module (e.g., 620 in FIG. 6), in effect, behaves as if a bandpass filter is inserted into the communication channel with the bandwidth of this bandpass filter varying with the thickness of the layer 50 of tissue; and a wireless RF transcutaneous link with digital modulation schemes (e.g., OOK modulated FSK modulation, PSK modulated OOK, etc.) that transfers power and control information between the external module (e.g., 610 in FIG. 6) and the implantable module (e.g., 620 in FIG. 6) is limited (in the context of typical practical circumstances) to 1 MHz bandwidth for an RF transmission frequency of about 5 MHz due to the thickness of the layer (e.g., 50 in FIG. 6) of tissue and from the quality factors of the primary and secondary LC-resonant tanks, such an RF link can suffer significant data integrity inconsistencies which can lead to audio degradation; and while the implantable module 620 can operate effectively under such conditions when implemented, e.g., using a complex ASIC, it would be advantageous if a different RF communication scheme could make use of a less complex implantable module practical.

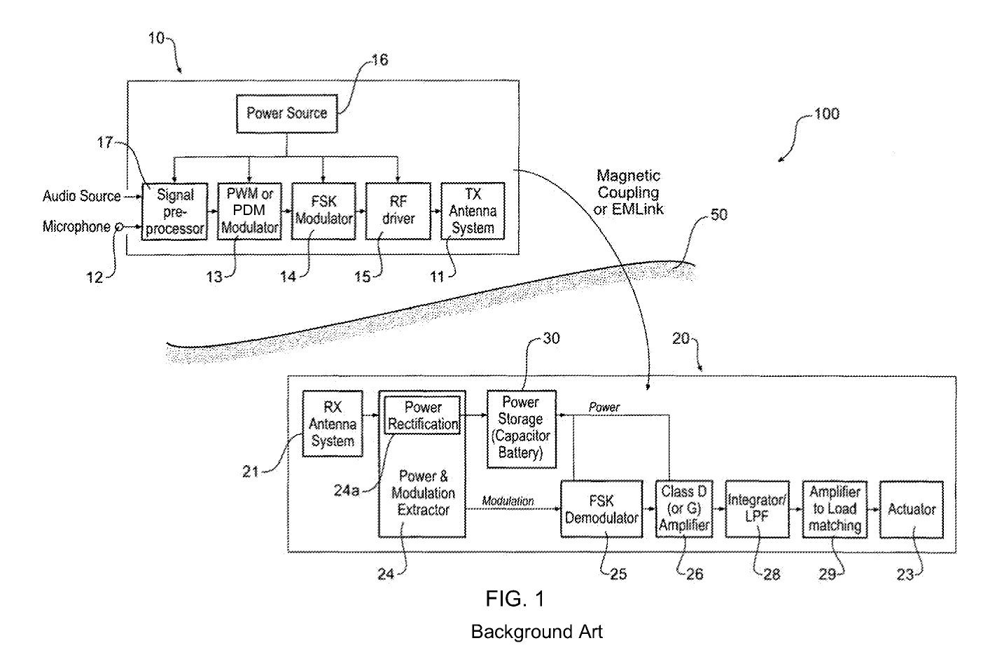

Furthermore, during the development of the present invention, among other things, the inventor recognized that aspects of FDS (frequency delta-sigma) modulation (FDSM) could be used to achieve a digital wireless RF, transcutaneous link between an external module and an implantable module of a medical implant system instead of the digital wireless RF link (OOK, FSK) of the Background Art. As such, in FIG. 6, the external module 610 includes (among other things) a frequency modulation (analog FM) modulator 613 but does not include a second, digital modulator, e.g., 14 in FIG. 1. Also as such, the implantable module 620 includes (among other things) a frequency-to-digital converter 633, but does not include a digital demodulator, e.g., 25 in FIG. 1.

Considered together, the operation of the FM modulator 613 and the frequency-to-digital converter 633 can be viewed as achieving a distributed variety of frequency delta-sigma (FDS) modulation (FDSM). The signal portion of the FDSM is performed by the FM modulator 613. The delta portion of the FDSM is performed by the frequency-to-digital converter.

In addition to the FM modulator 613, the external module 610 includes: a sound input unit 612 to receive sound signals; an optional pre-processor 617; an RF driver 615; a power source 616; and a coil 611 (e.g., included within a primary LC-resonant tank where, L represents the inductance of the coil and C the capacitance of, e.g., a series capacitor). The sound input unit 612 may be a component that receives an electronic signal indicative of sound, such as, for example, from an acoustic transducer such as a microphone or an external audio device. For example, sound input element 126 may receive a sound signal in the form of an electrical signal from an MP3 player electronically connected to sound input element 126. Alternatively, or in combination, the sound input unit 612 may be a test button or other user interface that the implantee or an operator may use to generate a test or other signal. In the case where the sound input unit 612 is an acoustic transducer, the transducer 612 converts the acoustic signal into a raw electrical signal. Connected to the transducer 612 is the optional pre-processor 617 (e.g., a conditioning amplifier), which pre-processes the raw electrical signal and outputs a pre-processed signal.

In addition to the frequency-to-digital converter 633 and the actuator 623, the implantable module 620 includes a coil 621 (e.g., included within a secondary LC-resonant tank, where L represents the inductance of the coil and C the capacitance of, e.g., a parallel capacitor), a power and modulation extractor unit 624 and a driver/amplifier 635. In FIG. 6, the coil 621 is not illustrated as being included within a housing of the implantable module 620. In other embodiments, the coil 621 may be provided within the housing of the implantable module, e.g., as indicated by the phantom boxes 620″ and 620′″. The coil 621 can be regarded as implantable because it is attached to the implantable module 620, and so it is implanted within the implantee, i.e., is implantable to the implantee, as contrasted with the coil 611 of the primary LC resonant tank that is external to the implantee. The implantable coil 621 inductively couples with, and so is energized by, the energized external coil 611, and thereby receives the amplified version of the pulse modulated signal. The implantable coil 621 transfers the amplified version of the pulse modulated signal to the power and modulation extractor unit 624.

Power to energize the frequency-to-digital converter 633 and the switched circuit 635 is extracted from the modulated signal by the power and modulation extractor unit 624. The power and modulation extractor unit 624 transfers a substantial equivalent to the frequency-modulated signal output by the FM modulator 613 to the frequency-to-digital converter 633. Optionally, the arrangement of the implantable module 620 may also include an audio pre-processing block (not illustrated in FIG. 6) for improving or optimizing the audio signal quality prior to the frequency-to-digital conversion. The power and modulation extractor unit 624 includes a rectification unit 624a (e.g., synchronous or diode half-wave/full-wave rectification).

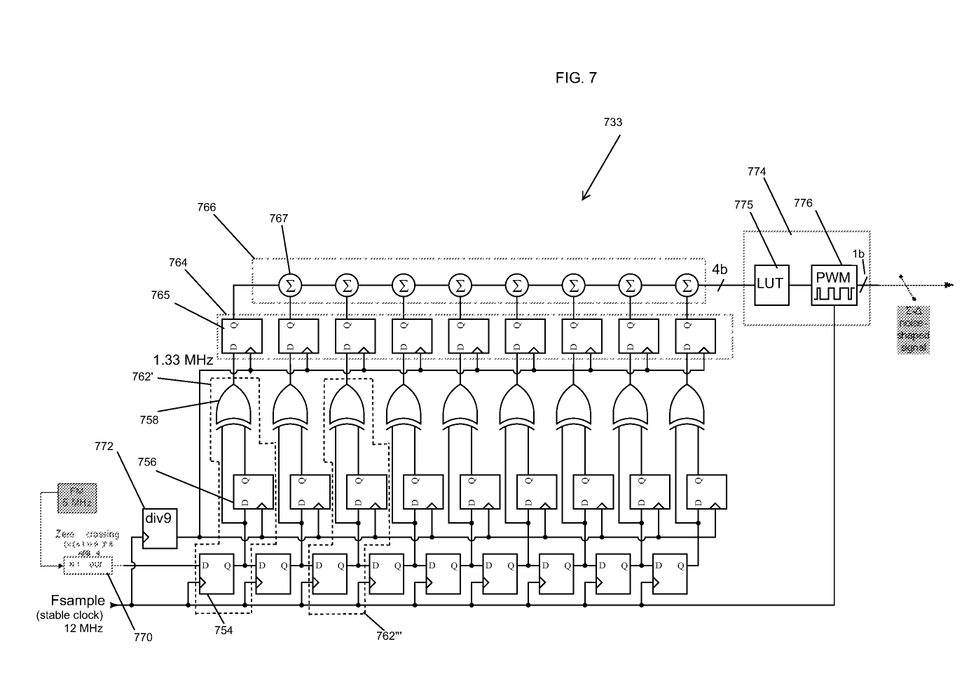

FIG. 7 illustrates an example of a narrow band frequency-to-digital converter 733 that can be used, e.g., as the frequency-to-digital converter 633, according to an embodiment of the present invention. The frequency-to-digital converter 733 can be described as a reduced sampling frequency type of frequency-to-digital converter. In general, the ‘reduced sampling frequency technique’ is understood by the skilled artisan, e.g., see the publication, Mats Hovin, Trond Saether, Dag T. Wisland, & Tor S. Lande, A Narrow-Band Delta-Sigma Frequency-To-Digital Converter, Proceedings of 1997 IEEE International Symposium on Circuits and Systems, Vol. 1, 77-80 (1997).

As a practical matter, when applying the ‘reduced sampling frequency technique’ to a frequency-to-digital converter, e.g., 733, the number of edges per second in the frequency-modulated signal that is subject to conversion by the frequency-to-digital converter 733 should not be evenly divisible by the sampling frequency at which the frequency-to-digital 733 operates. In FIG. 7, the frequency-to-digital 733 receives a stable clock signal with a frequency Fₛₐₘₚₗₑ, e.g., 12 MHz, and performs conversion upon a frequency-modulated signal having a frequency, F_{FM}, e.g., 5 MHz. Furthermore, the frequency-to-digital converter 733 is provided with a frequency-divider unit 772 that divides the sampling frequency, Fₛₐₘₚₗₑ by nine and provides the so-called reduced sampling frequency, F_{RS}, e.g., F_{RS}=1.33 MHz in FIG. 7. As a further example, if it is desired for the frequency-to-digital converter 733 to produce a 628 kbps bitstream, then the clock signal provided by the frequency-divider unit 772 typically will have a frequency of 628 kHz. The reduced sampling frequency F_{RS}, e.g., is about 8 times lower than the reduced sampling frequency F_{RS} is approximately F_{RS}=F_{FM}/8.

In light of the frequency-divider unit dividing the frequency Fₛₐₘₚₗₑ, by nine, the frequency-to-digital converter 733 is provided with nine cascaded instances of a building block whose first instance is 762′, which includes a first D-flip-flop 754, a second D-flip-flop 756 and an XOR (exclusive OR) gate 758. A third instance of the building block is called out as 762′″.

In the first instance of the building block 762′, the data input of the D-flip-flop 754 receives an output of a zero-crossing unit 770 (which itself has received the reconstructed frequency-modulated signal whose frequency is, e.g., 5 MHz). The zero-crossing block 770 is used to make a jump from the analog domain to the digital signaling domain. A non-inverted output (Q) of the D-flip-flop 754 is connected to a data input (D) of the D-flip-flop 756 and to a first input of the XOR gate 758. A non-inverted output (Q) of the D-flip-flop 758 is connected to a second input of the XOR gate 758. The clock input of the D-flip-flop 754 receives the sampling frequency, fₛₐₘₚₗₑ. A frequency-divider unit 772 divides the sampling frequency, fₛₐₘₚₗₑ by nine and provides the reduced frequency signal to the clock input of the D-flip-flop 756. An output of the XOR gate 758 is provided to a latch unit 764 that includes nine instances of a D-flip-flop 765. In particular, the output of the XOR gate 758 is provided to the data input of the first instance of the D-flip-flops 765 in the latch unit 764. The clock inputs of the nine instances of a D-flip-flop 765 also receive the reduced clock frequency from the frequency divider 772.

The non-inverted outputs of the nine instances of the D-flip-flop 765 are summed in a summation unit 766 that includes nine instances of an adder 767 to form a multi-bit bitstream of uniform pulse widths, e.g., a four parallel one-bit bitstreams at 1.33 MHz, that is provided to format-converter 774. The converter 774 includes a look-up table (LUT) 775 and a pulse-width modulator (PWM) 776. The converter 774 receives the 4-bit bitstream of uniform pulse widths and transforms it into a 1-bit bitstream of variable pulse widths. In effect, the converter 774 preserves the resolution of the 4-bit bitstream while converting it to a differently formatted bitstream.

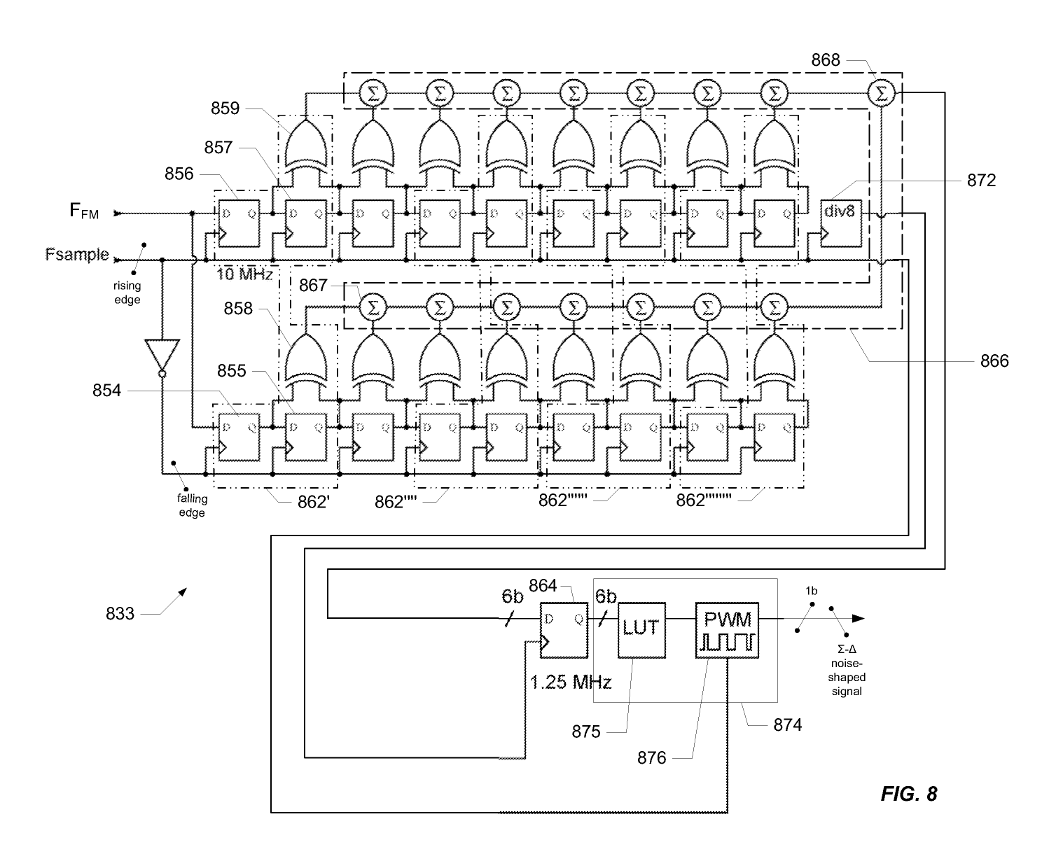

FIG. 8 illustrates an example of a wide band frequency-to-digital converter 833 that can be used, e.g., as the frequency-to-digital converter 633, according to an embodiment of the present invention. Whereas FIG. 7 illustrated a reduced-sampling reduced sampling frequency type of frequency-to-digital converter, by contrast, the frequency-to-digital converter 833 can be described as an oversampling type of frequency-to-digital converter.

For an oversampling type of frequency-to-digital converter, e.g., 833, again, the number of edges per second in the frequency-modulated signal that is subject to conversion by the frequency-to-digital converter 833 should not be evenly divisible by the sampling frequency at which the frequency-to-digital 833 operates. In FIG. 8, e.g., the frequency-modulation frequency F_{FM} is about 5.3 MHz and the sampling frequency, Fₛₐₘₚₗₑ is about 10 MHz or about 20,000,000 edges per second.

The frequency-to-digital converter 833 is provided with eight cascaded instances of a building block, of which the first, fourth, sixth and eighth instances are called out as 862′, 862″″, 862′″″ and 862″″″″, respectively. Taking the first instance 862′ as exemplary, it includes a first D-flip-flop 854 a second D-flip-flop 855, a third D-flip-flop 856, a fourth D-flip-flop 857, a first XOR (exclusive OR) gate 858 and a second XOR (exclusive OR) gate 859.

In the first instance of the building block 862′, the data inputs of the D-flip-flops 854 and 856 receive the reconstructed frequency-modulated. Non-inverted outputs (Q) of the D-flip-flops 854 and 856 are connected to data inputs (D) of the D-flip-flops 855 and 857, respectively, to first inputs of the XOR gates 858 and 859, respectively. Non-inverted outputs (Q) of the D-flip-flops 855 and 857 are connected to second inputs of the XOR gates 858 and 859, respectively.

The outputs of the eight instances of the XOR gate 858 and the outputs of the eight instances of the XOR gate 859 are summed in a summation unit 866 that includes seven instances of an adder 867. Seven instances of an adder 868 and one instance of an adder 869. The summation unit 866 produces a multi-bit bitstream of uniform pulse widths, e.g., a six parallel one-bit bitstreams at about 10 MHz. that is provided to a latch unit 864.

The frequency-to-digital converter 833 further includes a frequency-divider unit 872 and a format converter 874. The frequency divider 872 divides the sampling frequency, fₛₐₘₚₗₑ by eight and provides the reduced frequency signal to the clock input of the latch unit 864. The converter 874 includes a look-up table (LUT) 875 and a pulse-width modulator (PWM) 876. The converter 874 receives the 6-bit bitstream of uniform pulse widths and transforms it into a 1-bit bitstream of variable pulse widths. In effect, the converter 874 preserves the resolution of the 6-bit bitstream while converting it to a differently formatted bitstream.

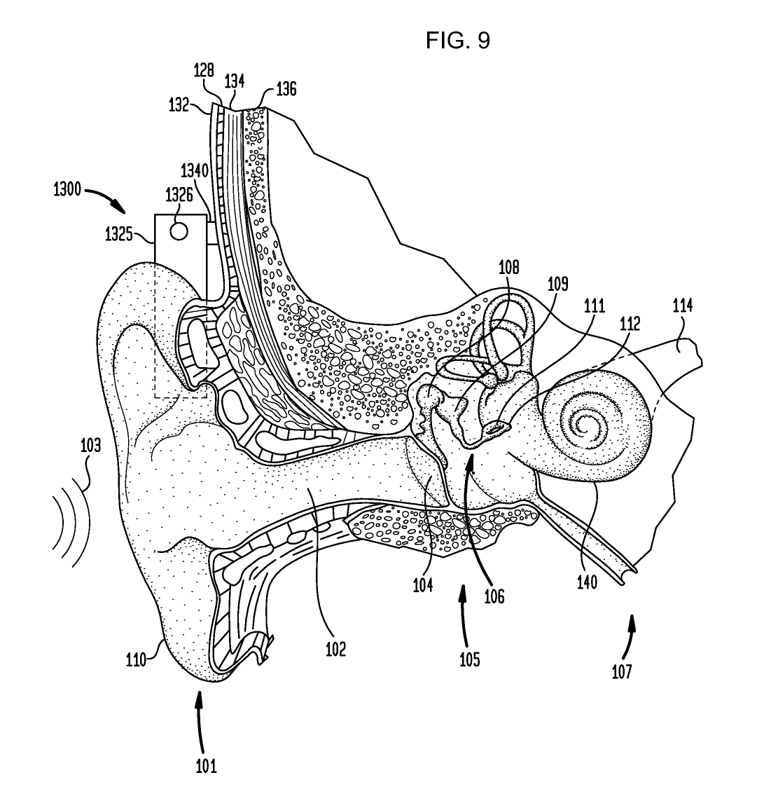

As noted above, embodiments of the present invention may also be used with other auditory prostheses. One other type of such auditory prosthesis that converts sound to mechanical stimulation in treating hearing loss is a bone conduction device. FIG. 9 is a perspective view of a bone conduction device 1300 in which embodiments of the present invention may be advantageously implemented. For ease of explanation, the portions of a recipient's outer ear 101, middle ear 105 and inner ear 107 are labeled with the same labels as used in FIG. 4. As will be discussed further below, bone conduction device 1300 converts a received sound signal into a mechanical force that is delivered to the recipient's skull.

FIG. 9 also illustrates the positioning of bone conduction device 1300 relative to outer ear 101, middle ear 105 and inner ear 107 of a recipient of device 1300. As shown, bone conduction device 1300 is positioned behind outer ear 101 of the recipient. In the embodiment illustrated in FIG. 9, bone conduction device 1300 comprises a housing 1325 having a sound input element 1326 positioned in, on or coupled to housing 1325. Sound input element 1326 is configured to receive sound signals and may comprise, for example, a microphone, telecoil, etc.

Bone conduction device 1300 comprises a sound processor, an actuator and/or various other electronic circuits/devices that facilitate operation of the device in the presently described embodiment. In an embodiment, the actuator is a piezoelectric actuator; however, in other embodiments, actuator can be any other suitable type actuator. Actuators are sometimes referred to as vibrators. Bone conduction device 1300 also comprises actuator drive components configured to generate and apply an electric field to the actuator. In certain embodiments, the actuator drive components comprise one or more linear amplifiers. For example, class D amplifiers or class G amplifiers may be utilized, in certain circumstances, with one or more passive filters. More particularly, sound signals are received by sound input element 1326 and converted to electrical signals. The electrical signals are processed and provided to the actuator that outputs a force for delivery to the recipient's skull to cause a hearing percept by the recipient.

Bone conduction device 1300 further includes a coupling 1340 configured to attach the device to the recipient. In the specific embodiments of FIG. 9, coupling 1340 is attached to an anchor system (not shown) implanted in the recipient. In the illustrative arrangement of FIG. 9, anchor system comprises a percutaneous abutment fixed to the recipient's skull bone 136. The abutment extends from bone 136 through muscle 134, fat 128 and skin 132 so that coupling 1340 can be attached thereto. Such a percutaneous abutment provides an attachment location for coupling 1340 that facilitates efficient transmission of mechanical force.

As noted, a bone conduction device, such as bone conduction device 1300, utilizes an actuator (also sometimes referred to as a vibrator) to generate a mechanical force for transmission to the recipient's skull. As with the above described DACs system, the bone conduction device 1300 uses the resonance peak(s) of the device in generating drive signals for generating the stimulation to be applied to the recipient in the presently described embodiment.

Housing 1325 includes a sound input element 1326, and may further include (not illustrated) a controller, a signal generator and an actuator. The controller is a circuit (e.g., an Application Specific Integrated Circuit (ASIC)) configured for exercising control over the bone conduction device. For example, the controller is configured for receiving, from the sound input element 1326, the sound signals and processing the sound signals to generate control signals for controlling signal generator in generating drive signals causing actuation of the actuator in the presently described embodiment. The controller takes into account the frequency response and resonant peak(s) of the actuator in determining the drive signals in the presently described embodiment. The actuator is any type of suitable transducer configured to receive electrical signals and generate mechanical motion in response to the electrical signals. For example, in an embodiment, the actuator is an electromagnetic actuator.

Embodiments of the present invention are described herein primarily in connection with two types of Active Implantable Medical Devices (AIMDs), namely DACS systems and bone conduction systems, but such embodiments are also applicable to cochlear implant systems (commonly referred to as cochlear prosthetic devices, cochlear prostheses, cochlear implants, cochlear devices, and the like; simply “cochlea implant systems” herein.) Cochlear implant systems generally refer to hearing prostheses that deliver electrical stimulation to the cochlea of a recipient. As used herein, cochlear implant systems also include hearing prostheses that deliver electrical stimulation in combination with other types of stimulation, such as acoustic or mechanical stimulation. It would be appreciated that embodiments of the present invention may be implemented in other types of AIMDs.

At least some of the embodiments described herein exhibit advantages including: simplicity of the implant electronics or ASIC; low implant component count results also in low power consumption; low distortion, independent of skin-flap thickness; use of an FM signal that has a constant envelope, such that signaling/information is available in the zero crossings of the phase, and thus can be amplified/buffered easily through class D, RF (e.g., 5 MHz) amplifiers; the pulse modulated signal has a substantially constant envelope and is substantially independent of the input signal (e.g., the electronic signal output by the acoustic transducer), thus voltage variations experienced by the implanted device are reduced if not minimized, and consequently power consumption is improved.

Various aspects of the present invention provide advantages over the Background Art. For example, the arrangement shown allows much of the circuit complexity to remain in the external module 10, with a simplified arrangement of the implantable module 20. The implantable circuitry is simplified in one form, e.g., by having the demodulator directly driving the amplifier. Furthermore, the arrangement does not require a separate PWM or PDM demodulator to remove the Pulse Width Modulation or Pulse Density Modulation of the original audio signal applied in the external module.

The arrangements described herein may be used in a uni-directional system (i.e. power and data flow from the external module to the implantable module) thus allowing for further simplification of the implantable module. The various aspects of the present invention have been described with reference to specific embodiments. It will be appreciated however, that various variations and modifications may be made within the broadest scope of the principles described herein.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

Reference herein to “one embodiment” or “an embodiment” means that a particular feature, structure, operation, or other characteristic described in connection with the embodiment may be included in at least one implementation of the present invention. However, the appearance of the phrase “in one embodiment” or “in an embodiment” in various places in the specification does not necessarily refer to the same embodiment. It is further envisioned that a skilled person could use any or all of the above embodiments in any compatible combination or permutation.

While various embodiments of the present invention have been described above, it should be understood that they have been presented by way of example only, and not limitation. It will be apparent to persons skilled in the relevant art that various changes in form and detail may be made therein without departing from the scope of the invention. Thus, the breadth and scope of the present invention should not be limited by any of the above-described exemplary embodiments, but should be defined only in accordance with the following claims and their equivalents.

What is claimed is:

1. In an active implantable medical device (AIMD) including an external module and an implantable module having a stimulation transducer implantable in an implantee and configured to deliver stimulation energy to auditory tissue so as to cause a hearing percept, the method comprising:
receiving, at the implantable module, from the external module via a transcutaneous RF link, an analog frequency-modulated RF signal (analog FM) including stimulation signals representative of sound;
performing frequency-to-digital conversion upon the frequency-modulated signal to obtain pulse-formatted signals corresponding to the stimulation signals; and
energizing the stimulation transducer based upon the pulse-formatted signals to cause the hearing percept.

2. The method of claim 1, wherein the step of performing frequency-to-digital conversion includes:
generating a sigma-delta modulated stream of pulses based upon the frequency-modulated RF signal; and
filtering the pulses.

3. The method of claim 1, wherein the pulse-formatted signals are one of pulse width modulated signals and pulse density modulated signals.

4. The method of claim 1, wherein the step of performing frequency-to-digital conversion includes:
sampling the frequency-modulated signal at one of a reduced sampling frequency, F_{RS}, and an oversampling frequency.

5. The method of claim 4, wherein:
the frequency-modulated RF signal has a carrier frequency; and
the carrier frequency, F_{C}, is not an even integer multiple of a sampling frequency.

6. The method of claim 1, wherein the stimulation signals are transferred over the transcutaneous inductive RF link by magnetically coupling between an external antenna coil and an implanted antenna coil.

7. The method of claim 6, wherein the step of receiving further includes:
extracting a power signal-component from the received RF signal; and
using the power signal-component to supply energy to one or more parts of the implantable module.

8. The method of claim 1, wherein:
the implantable module is sealed in a biocompatible casing material.

9. An implantable module of an active implantable medical device (AIMD) implantable in an implantee, the implantable module comprising:
an antenna to receive an analog frequency-modulated signal including stimulation signals representative of sound,
a frequency-to-digital converter operable upon the frequency-modulated signal to obtain pulse-modulated signals;
a driver circuit responsive to the frequency-to-digital converter; and
a stimulation transducer responsive to the driver circuit;
the driver circuit being configured to energize the stimulation transducer based upon the pulse-formatted signals; and
the stimulation transducer being configured to deliver stimulation energy to auditory tissue based upon stimulation signals so as to cause a hearing percept.

10. The implantable module of claim 9, wherein the frequency-to-digital converter is further operable to:
generate a sigma-delta modulated stream of pulses based upon the frequency-modulated RF signal; and
filter the pulses.

11. The implantable module of claim 9, wherein the frequency-to-digital converter is further operable to convert the received frequency-modulated signal into one of a pulse width modulated signal and a pulse density modulated signal.

12. The implantable module of claim 9, wherein the frequency-to-digital converter is further operable to sample the frequency-modulated signal at a reduced sampling frequency.

13. The implantable module of claim 12, wherein the frequency-to-digital converter includes multiple cascaded instances of a building block that includes:
an exclusive-OR (XOR) gate; and
first and second flip-flops that provide latched data, respectively, to the XOR gate.

14. The implantable module of claim 13, wherein the frequency-to-digital converter further includes:
a summation device that receives outputs of the multiple instances of the XOR gate and outputs a multi-bit bitstream of uniform pulse widths; and
a format converter arranged to receive an output of the summation device and to produce a 1-bit bitstream of non-uniform pulse widths corresponding to the multi-bit bitstream of uniform pulse widths.

15. The implantable module of claim 9, wherein the frequency-to-digital converter is further operable to sample the frequency-modulated signal at an oversampling frequency.

16. The implantable module of claim 14, wherein the frequency-to-digital converter includes multiple cascaded instances of a building block that includes:
a first exclusive-OR (XOR) gate;
first and second flip-flops that provide latched data, respectively, to the first XOR gate;
a second exclusive-OR (XOR) gate; and
third and fourth flip-flops that provide latched data, respectively, to the second XOR gate, respectively.

17. The implantable module of claim 13, wherein the frequency-to-digital converter further includes:
a summation device that receives outputs of the multiple instances of the XOR gate and outputs a multi-bit bitstream of uniform pulse widths;
a latch unit to delay the multi-bit bitstream; and
a format converter arranged to receive an output of the latch unit and to produce a 1-bit bitstream of non-uniform pulse widths corresponding to the multi-bit bitstream of uniform pulse widths.

18. The implantable module of claim 9, wherein implantable module further includes:
a power and modulation extractor operable upon a signal from the antenna to extract a power component therefrom and to supply energy to at least the frequency-to-digital converter and the driver circuit.

19. In an active implantable medical device (AIMD) including an external module and an implantable module having a stimulation transducer implantable in an implantee and configured to deliver stimulation energy to auditory tissue so as to cause a hearing percept, the method comprising:
performing, at the external module, analog frequency-modulation (analog FM) upon sound signals;
receiving, at the implantable module, from the external module via a transcutaneous RF link, a frequency-modulated RF signal including stimulation signals representative of sound;
performing frequency-to-digital conversion upon the frequency-modulated signal to obtain pulse-formatted signals corresponding to the stimulation signals; and
energizing the stimulation transducer based upon the pulse-formatted signals to cause the hearing percept; and
wherein, taken together, the frequency modulation and the frequency-to-digital conversion represent a distributed form of frequency delta-sigma (FDS) modulation (FDSM).

20. An implantable module of an active implantable medical device (AIMD) implantable in an implantee, the implantable module comprising:
an analog frequency-modulation modulator to produce frequency-modulated signals representing sound signals;
a first antenna to transmit a radio frequency (RF) signal including the frequency-modulated signals;
a second antenna to receive a frequency-modulated RF signal;
a frequency-to-digital converter operable upon the frequency-modulated RF signal to obtain pulse-formatted signals;
a driver circuit responsive to the frequency-to-digital converter; and
a stimulation transducer responsive to the driver circuit;
the driver circuit being configured to energize the stimulation transducer based upon the pulse-formatted signals; and
the stimulation transducer being configured to deliver stimulation energy to auditory tissue based upon stimulation signals so as to cause a hearing percept; and.
wherein, taken together, the frequency modulation and the frequency-to-digital conversion represent a distributed form of frequency delta-sigma (FDS) modulation (FDSM).
